(19) United States
(12) Patent Application Publication
(10) Pub. No.: US 2020/0016215 A1
(43) Pub. Date: Jan. 16, 2020

(54) BIOACTIVE MUSSEL COMPOSITIONS AND/OR EXTRACTS

(71) Applicant: SANFORD LIMITED, Freemans Bay Auckland (NZ)

(72) Inventor: Hong (Sabrina) TIAN, Freemans Bay Auckland (NZ)

(73) Assignee: SANFORD LIMITED, Freemans Bay Auckland (NZ)

(21) Appl. No.: 16/472,020

(22) PCT Filed: Dec. 20, 2017
(86) PCT No.: PCT/NZ2017/050166

§ 371 Date: Jun. 20, 2019

(30) Foreign Application Priority Data
Dec. 20, 2016 (NZ) 727786

(51) Int. Cl. A61K 35/618; A23L 17/50; A23L 33/18; A61K 9/107

(52) U.S. Cl. CPC A61K 35/618; A23L 17/50; A23V 2002/00; A61K 9/107; A23L 33/18

(57) ABSTRACT

The invention relates to a liquid or dried composition derived from mussels having a high yield of bioactive components with improved bioavailability. The compositions have at least two phases, including at least one hydrophobic phase having one or more lipid and/or lipophilic bioactive compounds, and one or more hydrophilic or aqueous phases having one or more bioactive components dispersed or suspended therein. The liquid composition has an emulsion-like structure and the dried composition has the properties of a self-emulsifying composition when rehydrated. The compositions are naturally stable and include uniformly dispersed spherical shaped biomaterials with a majority of particles sizes of between about 100 nm-50,000 nm. The structure of the compositions provides improved bioavailability of bioactive compounds and a wide variety of formulation options.

### FIELD OF INVENTION

The present invention relates generally to compositions or extracts derived from mussels comprising a high yield of bioactive components with improved bioavailability.

### BACKGROUND OF INVENTION

Shellfish, including mussels have long been part of the diet of human populations. Extensive research has been conducted in relation to the health benefits and bioactive properties of mussels. For example, the unique properties of the New Zealand green-lipped mussel (Perna canaliculus) have been studied for more than 40 years. It was observed that New Zealand coastal Maori populations historically had lower incidences of arthritis than inland Maori populations. This was attributed to the high consumption of green-lipped mussels by the coastal Maori populations thereby suggesting that the green-lipped mussel species had anti-inflammatory activity. Clinical trials have shown that lipid extracts of Perna canaliculus do have anti-inflammatory activity and can be used in the management of arthritis (Halpern (2000) Anti-inflammatory effects of a stabilized lipid extract of Perna canaliculus (Lyprinol); Brien et al. (2008) Systematic review of the nutritional supplement Perna Canaliculus (green-lipped mussel) in the treatment of osteoarthritis Q J Med 2008; 101:167-179). Various types of green-lipped mussel lipid extracts have been commercialised for use in the relief of arthritic symptoms, and research has generally been focused in this area.

Mussels such Perna canaliculus typically contain high levels of Omega-3 fatty acids and they are a rich source of other beneficial bioactive components including vitamins, minerals, taurine, amino acids, polyphenols, carotenoids and active compounds of glucosaminoglycan (GAG or mucopolysaccharide), collagen and glycogen, some of which have been shown to have positive health effects (Grienke et al. (2014) Bioactive compounds from marine mussels and their effects on human health Food Chemistry 142 (2014) 48-60; Coulson et al and Rainsford et al (2015) Novel Natural Products: Therapeutic Effects in Pain, Arthritis and Gastro-intestinal Diseases, Progress in Drug Research 70). Until recently there has been little emphasis or research into the potential health properties of non-lipid components present in whole mussel compositions. It is common for the non-lipid components to be discarded or used as low value by-products after recovery of the lipid fraction.

The structure and biochemical composition and/or properties of mussel compositions or extracts is influenced by the processing methods used to make the compositions or extracts. It is very challenging to extract the beneficial bioactive components from mussels because it can be difficult to open the shells to remove or separate the meat and other biological material from inside the shells, in a manner which preserves the nature and quantity of bioactive components present in the meat and other biological material. Conventional processing methods to remove the material from inside the shells typically involve mechanical processing such as machine shucking, crushing, or grinding to open or break the shells, or the use of heat which damages bioactive components. Once the shells are opened and the flesh is accessible or extracted, mechanical techniques are used to comminute the mussel flesh, for example, homogenising, blending, grinding, mincing, milling, pulverising or centrifuging the flesh. This is usually followed by low temperature drying, such as freeze drying of the comminuted material. Mechanical processes such as these generally yield poorly soluble extracts as the biological materials are not fully broken down to release their functional components. The compositions are unstable and typically have large irregular shaped and sized particles with non-uniform distribution. Anti-oxidants and other chemical additives are required to be added to the compositions to stabilise the compositions and to maintain any bioactivity that might be present in the compositions. In these processes, the mussel starting material is usually altered significantly as a result of the processing steps which can eliminate, destroy or change bioactive compounds present in the original mussel material thereby affecting the bioactive properties.

Existing whole mussel powders and mussel lipid extracts are generally poorly soluble in aqueous media (including the digestive tract) because of their large particle structure and their hydrophobic properties. They therefore present considerable formulation challenges and often suffer from poor or irregular bioavailability when given orally or via other routes requiring transmembrane absorption.

It is difficult to achieve consistent end-products having reliable and consistent bioavailability and thus efficacy of bioactive compounds.

Furthermore, existing whole mussel compositions and lipid extracts are known to have an unpleasant taste profile which makes them undesirable for formulation into some product formats and poses further formulation challenges.

### OBJECT OF THE INVENTION

It is an object of the invention to provide an improved bioactive mussel composition or extract that ameliorates some of the disadvantages and limitations of the known art, or at least provides the public with a useful choice.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

Further aspects and advantages of the present invention will become apparent from the ensuing description which is given by way of example only.

### SUMMARY OF INVENTION

In one aspect the invention relates to a whole mussel composition having at least two phases, including at least one hydrophobic phase comprising one or more lipid and/or lipophilic bioactive compounds, and one or more hydrophilic or aqueous phases having one or more bioactive components dispersed or suspended therein.

Preferably the mussel composition is in the form of a liquid composition comprising an emulsion-like structure wherein the at least one hydrophobic phase comprises one or more lipid and/or lipophilic bioactive compounds, and said one or more lipid and/or lipophilic bioactive compounds are dispersed in said hydrophilic or aqueous phase, and wherein said hydrophilic or aqueous phase has one or more hydrophilic bioactive components dispersed or suspended therein.

Preferably the hydrophilic bioactive components include one or more of proteins, peptides, amino acids, carbohydrates, vitamins, elements, glycogens, polysaccharides, minerals, taurine, polyphenols, carotenoids, glucosaminoglycan and collagen.

Preferably the hydrophobic phase comprises at least some droplets or globules having a layer surrounding or encapsulating the droplets or globules wherein the lipid or lipophilic bioactive components are located inside the droplets or globules.

The particles or globules may be lipoproteins or similar.

The composition may also comprise some solid particles in suspension.

Preferably the composition comprises a mixture of particle sizes, with a mean particle size distribution of between about 100 nm-100 μm, including some micro-particles and/or micro-droplets and/or some nano-particles and/or nano-droplets.

Preferably the majority of the particles in the composition are micro-particles with sizes in the range of between 100-50,000 nm.

Preferably the composition comprises at least 5% lipid or lipophilic components in the hydrophobic phase, and at least 75% hydrophilic components in the hydrophilic phase.

Preferably the composition comprises between about 5-20% lipid or lipophilic components in the hydrophobic phase.

Preferably the composition comprises one or more natural components functioning as emulsifiers, surfactants and/or co-surfactants.

Preferably the composition comprises at least a total of 5% of one or more surfactants or surfactant-like, emulsifiers or emulsifier-like, co-surfactant or co-surfactant-like components.

Preferably the one or more natural components functioning as emulsifiers, surfactants and/or co-surfactants are proteins and/or peptides dispersed in the hydrophilic phase, and/or located on the surface of particles or globules in the hydrophobic phase.

Preferably the composition is a hydrolysed mussel composition produced by enzyme hydrolysis.

Preferably no additional chemical surfactants and/or co-surfactants and/or emulsifying agents are included in the mussel composition.

Preferably the composition is stable without the addition of any anti-oxidants.

Preferably the composition is dried to form a dried composition.

Preferably the composition is a freeze dried composition.

Preferably the dried composition when rehydrated in water or aqueous media forms a liquid composition with the properties of the liquid composition as described herein.

Preferably the dried composition has the properties and/or characteristics of a self-emulsifying composition.

Preferably the composition has a natural self-emulsifying drug delivery system (SEDDS)-like structure or a bioactive delivery system-like structure, which provides improved bioavailability of the bioactive components present in the composition.

Preferably the composition is formulated into a dietary supplement composition, a nutraceutical composition, a veterinary composition or a pharmaceutical composition.

Preferably the composition is formulated into a dosage form selected from the group comprising oral dosage forms such as tablets, capsules, dried powder formats, oils, food ingredients; topical dosage forms for external use such as creams, gels, emollients, ointments, lotions, dressings such as plasters and bandages and medicated dressings; and other internal dosage forms including injectable forms

In a further aspect of the invention there is provided a dried self-emulsifying composition and/or bioactive delivery system derived from mussels, said composition having at least two phases, including at least one hydrophobic phase comprising one or more lipid and/or lipophilic bioactive compounds, and at least one hydrophilic or aqueous phase having one or more bioactive components dispersed or suspended therein, and one or more natural components functioning as surfactants and/or co-surfactants and/or emulsifiers, such that on addition to an aqueous media the composition forms an emulsion-like structure.

Preferably the composition is a hydrolysed mussel composition produced by enzyme hydrolysis.

Preferably the one or more natural components functioning as surfactants and/or co-surfactants and/or emulsifiers are proteins and/or peptides dispersed in the hydrophilic phase, and/or located on the surface of particles and/or globules in the hydrophobic phase.

In a further aspect the invention relates to a lipid mussel extract produced from a liquid or dried mussel composition as described herein by extraction or fractionation and recovery of the hydrophobic phase of the mussel composition.

Preferably the lipid mussel extract is an encapsulated oil.

In a further aspect the invention relates to a non-lipid mussel extract produced from a liquid or dried mussel composition as described herein by extraction or fractionation and recovery of the hydrophilic phase of the mussel composition.

According to a further aspect of the invention there is provided a method of preparing a mussel composition as described herein from mussels, wherein the method comprises at least one enzyme treatment step applied to uncomminuted mussel material.

Preferably the enzyme treatment step is applied to whole fresh mussels.

Preferably the enzyme treatment step comprises:
gapping or at least partially opening the shells of the mussels or otherwise exposing at least some of the material inside the shells of the mussels;
applying an enzyme formulation comprising one or more enzymes to the mussels, and
   (a) leaving the mussels in contact with the said enzyme formulation until the flesh and other biological material is substantially separated from the shells of the mussels, and
   (b) liquefying the flesh and other biological material by the use of the same enzyme formulation in the same enzyme treatment step, and/or by applying a different enzyme formulation in the same or one or more subsequent enzyme treatment steps.

Preferably the enzyme formulation in step (a) includes at least one proteolytic enzyme.

Preferably the mussels are whole live mussels at or up to the point of the gapping or opening step.

Preferably the duration of the or each enzyme treatment step is less than 120 minutes, more preferably less than 90 minutes and even more preferably is in the range of 15-40 minutes.

Preferably the mussels are selected from the following species: New Zealand green-lipped mussel (Perna canaliculus), the Asian green mussel (Perna viridis), other Perna species; the Mediterranean blue mussel (Mytilus galloprovincialis), the common blue mussel (Mytilus edulus), California mussel (Mytilus californianus), the brown mussel (Perna perna), the Korean mussel (Mytilus coruscus), the Chilean mussel (Mytilus chilensis), the bay mussel (Mytilus trossulus), the ribbed mussel (Geukensia demissa), the date mussel (Lithophaga lithophaga), the fresh water Zebra Mussel (Dreissena polymorpha); Brachidontes rodriguezii; Perumytilus purpuratus; Aulacomya ater; Choromytilus chorus; and Modiolus species.

Preferably the dried composition has high solubility in aqueous media and can be rehydrated to form a stable emulsion-like liquid composition.

Preferably the liquid and/or dried shellfish composition comprises a high yield of bioactive components or concentrated active ingredients.

This invention may also broadly be said to consist in the parts, elements and features referred to or indicated in the specification of the application, individually or collectively, and any or all combinations of any two or more of the parts, elements or features, and where specific integers are mentioned herein which have known equivalents such equivalents are deemed to be incorporated herein as if individually set forth.

Definitions

In this specification, unless the context otherwise requires, the following terms shall have the following definitions:

“whole” as used herein in connection with mussels means mussels including their shells that are substantially whole and intact, and substantially unprocessed.

“fresh” as used herein in connection with mussels means mussels that are alive or have died less than 12 hours before processing commences, but preferably less than three hours before processing commences.

“bioactive components” and/or “bioactive compounds” (used interchangeably) means any one or more chemical molecules, elements or compounds that has an effect on a living organism, tissue or cell or gene, and includes any molecule(s), element(s) or compound(s) or combinations thereof that are or may be beneficial to the health or wellbeing of humans and other animals.

“emulsion-like composition” in the context of the invention means a liquid composition resembling an emulsion or a colloid, comprising at least one hydrophilic phase or continuous phase and at least one hydrophobic phase or dispersed phase, and which may also comprise some solid particles in solution or suspension. The term includes colloidal suspensions, colloidal emulsions and colloidal dispersions.

“emulsion” includes all types of emulsions, including macro-emulsions, single emulsions, double emulsions, multiple emulsions, micro-emulsions, nano-emulsions, colloidal emulsions and emulsified suspensions.

“enzyme formulation” means a formulation comprising at least one enzyme and includes formulations comprising a mixture of one or more enzymes, and formulations comprising one or more enzymes and one or more other non-enzyme substances.

“self-emulsifying” as used herein refers to compositions which spontaneously or with only minimal agitation form a stable emulsion or dispersion when added to an aqueous medium.

“stable” as used herein in connection with the emulsion-like compositions of the invention refers to liquid compositions or rehydrated dried compositions which exhibit no phase separation when kept, without agitation, at room temperature for one hour or longer.

“target biological material” or “target substrates” as used herein refers to any desired biological material present on or in the shells of the mussels, namely the meat or flesh inside the shells, but also including other material such as chitosan present on the shells, layers of biological material that might be present inside the shells (for example, the nacre, prismatic and periostracum layers present in mussels (resembling skin)), ligaments, abductor muscles, teeth, byssus threads (or beards), gut and feet.

### DESCRIPTION

The invention will now be described, by way of example only, with reference to the accompanying drawings:

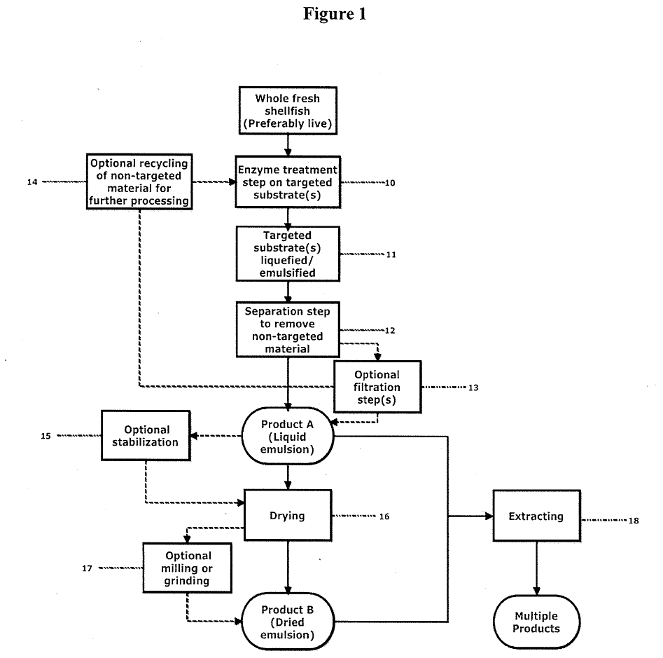

FIG. 1 is a flow chart of a preferred process for producing a liquid or dried composition of the invention from mussels.

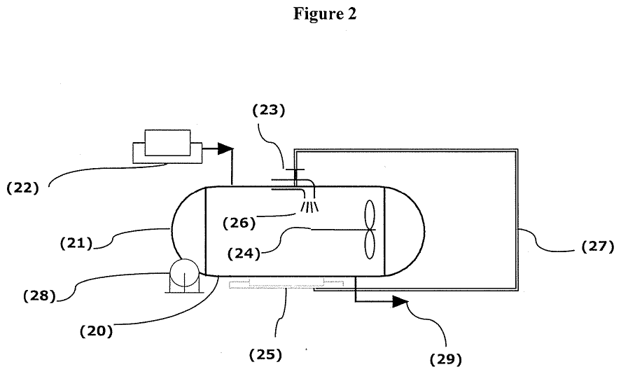

FIG. 2 is a schematic representation of a treatment chamber that may be used in the process of FIG. 1.

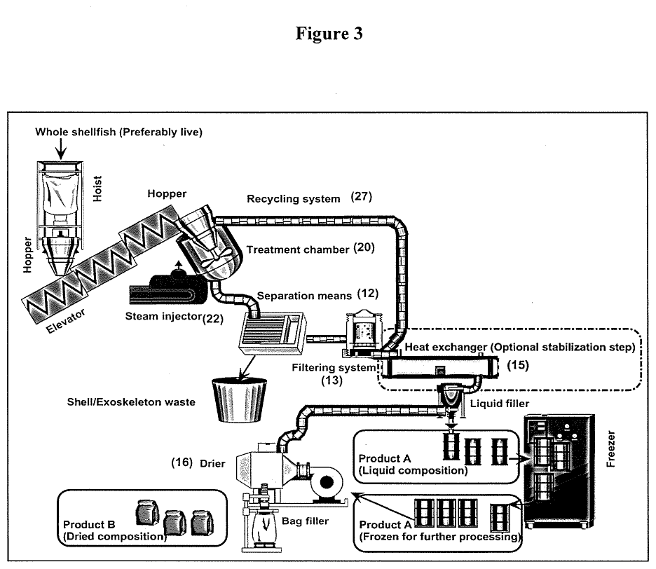

FIG. 3 is a schematic representation of a preferred process for producing a liquid or dried composition from mussels incorporating the treatment chamber of FIG. 2.

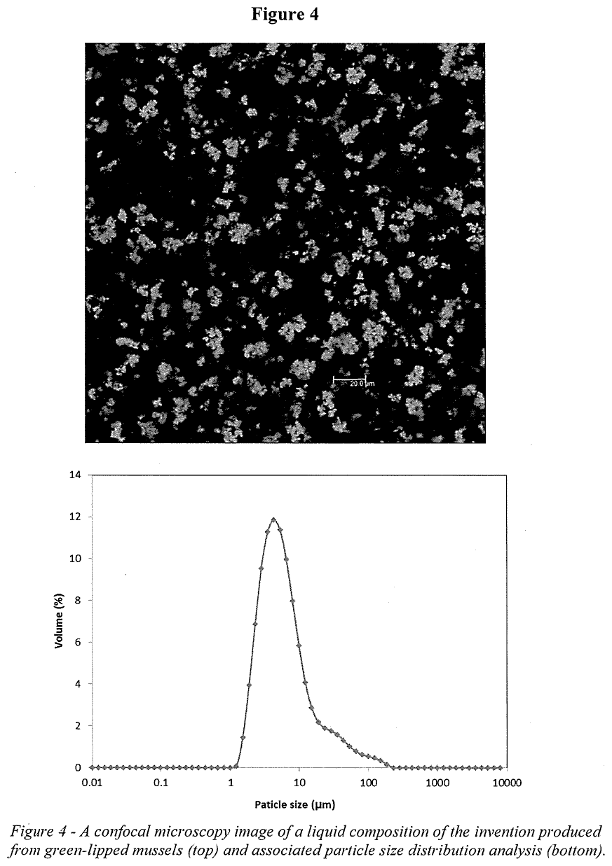

FIG. 4 is a confocal microscopy image and associated graph showing the particle size distribution of a liquid composition of the invention produced from green-lipped mussels.

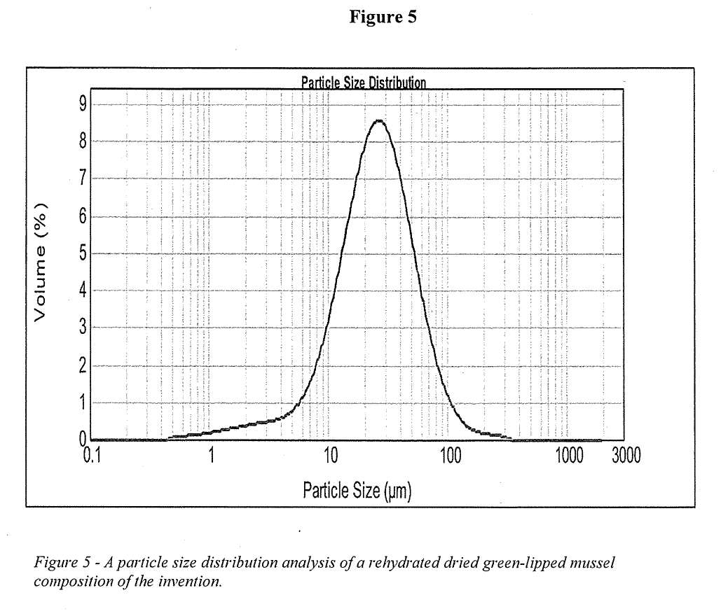

FIG. 5 is a graph showing the particle size distribution of a rehydrated dried composition of the invention produced from green-lipped mussels.

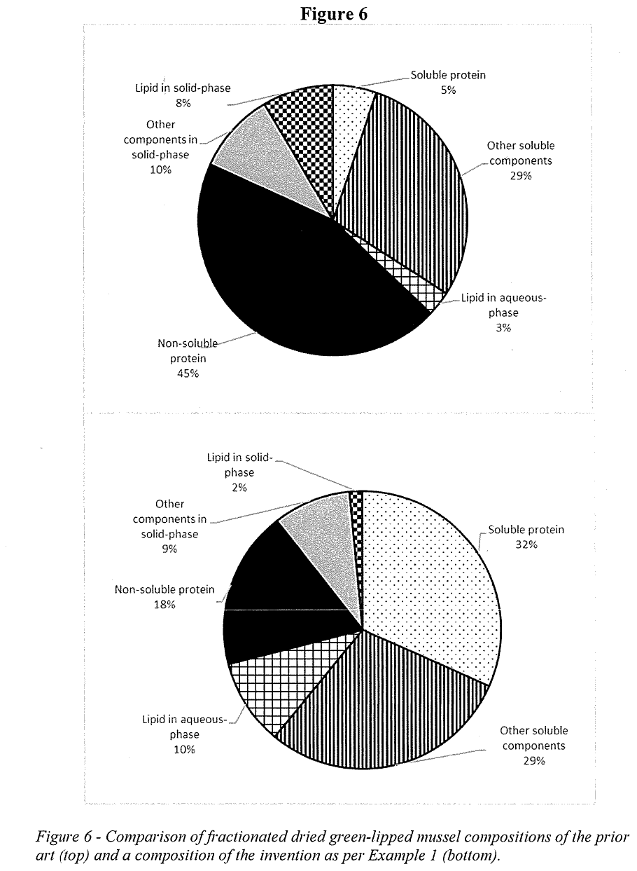

FIG. 6 is a graph showing a comparison of the fractions of a dried green-lipped mussel composition of the invention produced by enzyme hydrolysis (shown on the right) with the fractions of a typical dried green-lipped mussel composition produced by mechanical processing methods (shown on the left).

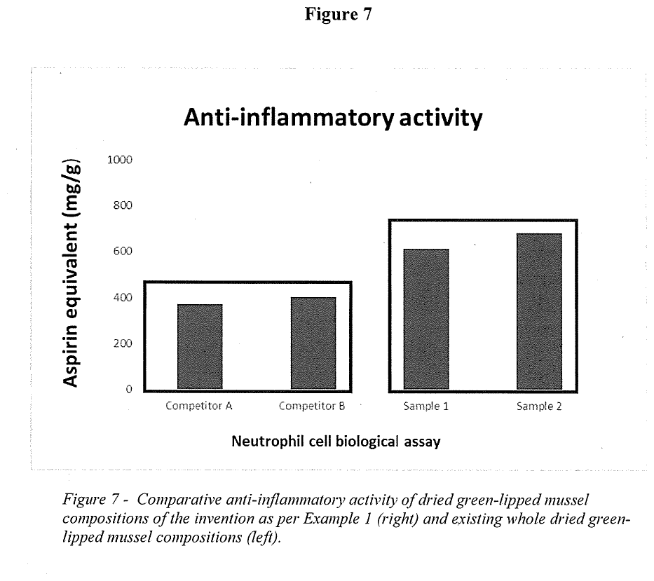

FIG. 7 is a graph showing the results of an anti-inflammatory activity study of two dried green-lipped mussel compositions of Example 1 in comparison with the anti-inflammatory activity of two existing dried green-lipped mussel products.

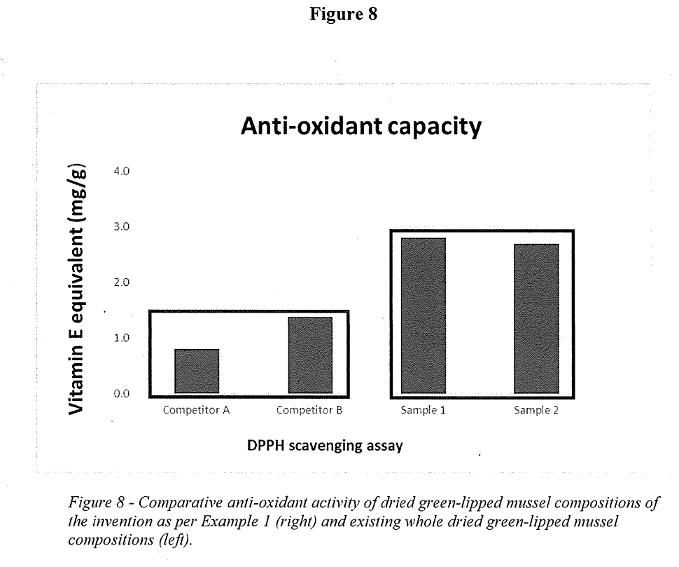

FIG. 8 is a graph showing the results of an antioxidant activity study of two dried green-lipped mussel compositions of Example 1 in comparison with the antioxidant activity of two existing dried green-lipped mussel products.

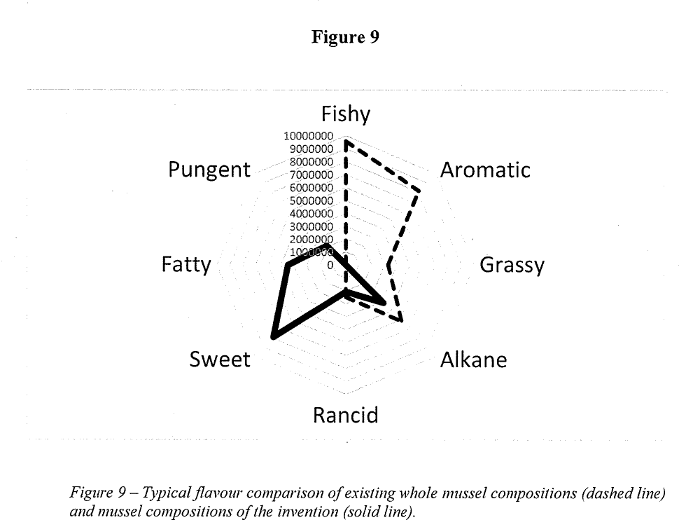

FIG. 9 is a graph showing the typical flavour profile of a dried green-lipped mussel composition of the invention compared to existing whole mussel composition products.

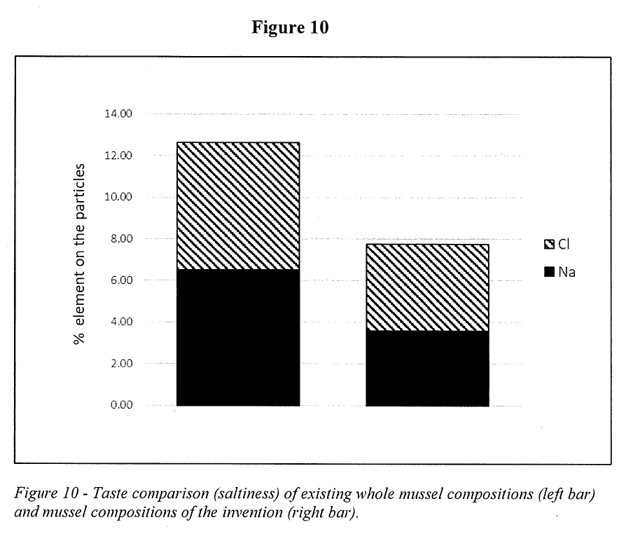

FIG. 10 is a graph showing the typical taste profile (saltiness) of a dried green-lipped mussel composition of the invention compared to existing whole mussel composition products.

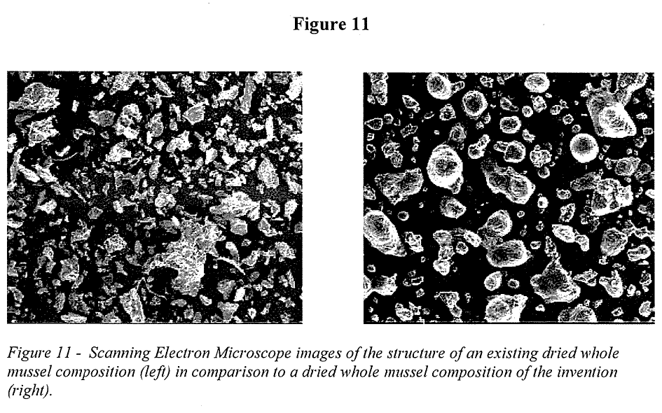

FIG. 11 are some scanning electron microscope (SEM) images collected on FEI Quanta 450 SEM showing the microstructure of a dried green-lipped mussel composition of the invention compared to the microstructure of an existing whole mussel composition product. Samples were fixed on SEM stages and coated with carbon to optimise visualisation.

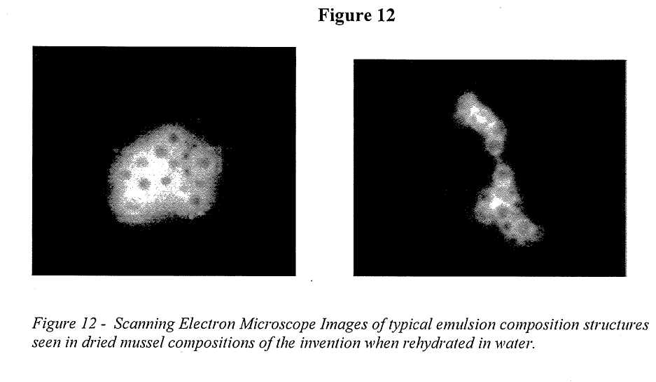

FIG. 12 are some SEM images showing the typical microstructure of dried green-lipped mussel composition of the invention rehydrated in water.

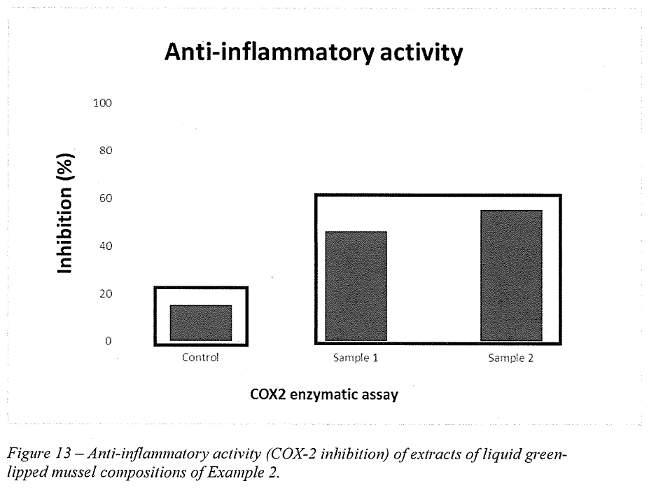

FIG. 13 is a graph showing the results of an anti-inflammatory activity (COX-2 inhibition) assay of two dried green-lipped mussel compositions of Example 2.

The following description will describe the invention in relation to preferred embodiments of the invention, however the invention is in no way limited to these preferred embodiments as they are purely to exemplify the invention only and it is envisaged that possible variations and modifications could be made that would be readily apparent to those skilled in the art without departing from the scope of the invention.

The invention relates to liquid or dried compositions derived from mussels comprising a high concentration or high yield of bioactive components, and improved bioavailability. The invention is directed particularly, but not necessarily solely, towards compositions derived from the processing of whole fresh or live mussel species including, but not limited to the following: all mussel species such as the New Zealand green-lipped mussel (Perna canaliculus), the Asian green mussel (Perna viridis), other Perna species; the Mediterranean blue mussel (Mytilus galloprovincialis), the common blue mussel (Mytilus edulus), California mussel (Mytilus californianus), the brown mussel (Perna perna), the Korean mussel (Mytilus coruscus), the Chilean mussel (Mytilus chilensis), the bay mussel (Mytilus trossulus), the ribbed mussel (Geukensia demissa), the date mussel (Lithophaga lithophaga), the fresh water Zebra Mussel (Dreissena polymorpha), Brachidontes rodriguezii, Perumytilus purpuratus, Aulacomya ater, Choromytilus chorus, and Modiolus species.

The compositions have an emulsion-like structure when in liquid form, and when dried and rehydrated with aqueous media. The compositions appear to have the properties of a self-emulsifying composition. The property of self-emulsification permits such compositions to be administered in concentrated form, as for example in oral dosage forms such as tablets and soft-gel or hard-shell capsules with the expectation that a fine emulsion will be formed in the digestive tract, so that when given orally, or by other administration methods, there is improved absorption of bioactive compounds. Self-emulsifying compositions when combined with an aqueous medium have improved physical stability when compared with conventional emulsions. Independent tests have been done on the compositions of the invention which show that the compositions are stable and exhibit little phase separation when left for long periods of time.

The compositions appear to spontaneously self-emulsify upon addition to water or other aqueous media. This surprising and beneficial property of the compositions enables the delivery of bioactive components in a form which, due to the stability and homogeneity of the resulting emulsion-like compositions, will provide good and unexpectedly consistent bioavailability.

The emulsion-like composition is a stable composition having at least two phases, namely a continuous phase and a dispersed phase, wherein at least one phase is a hydrophobic phase and at least one phase is a hydrophilic or aqueous phase, and the composition may also comprise some solid particles in solution and/or suspension. Preferably the composition comprises a mixture of particle sizes, with a mean particle size distribution of between 100 nm-100 μm, and including some micro-particles and/or micro-droplets and/or some nano-particles and/or nano-droplets. It has been found that the majority of the particles in the compositions of the invention are micro-particles with sizes in the range of between 100-50,000 nm. It has been found that at least some of the particles in the hydrophobic phase have a layer encapsulating or surrounding the particles or droplets or globules wherein one or more lipid or lipophilic bioactive compounds are located inside the particles or droplets or globules and are protected. The particles or globules may be lipoproteins or similar. The hydrophobic phase is dispersed and/or suspended in the continuous or hydrophilic or aqueous phase. The continuous or hydrophilic or aqueous phase has one or more bioactive components dispersed and/or suspended therein, which may include proteins, peptides, amino acids, carbohydrates, vitamins, elements, glycogens, polysaccharides, minerals, taurine, polyphenols, carotenoids, glucosaminoglycan and collagen.

The emulsion-like compositions are stable in either liquid form or dried form, and they comprise uniformly distributed spherical shaped particles, droplets and/or globules or biological molecules of reasonably uniform size and shape (as shown in FIGS. 4 and 5 relating to Example 1 below and in FIGS. 11 and 12). The compositions and any subsequent extracts produced therefrom have been shown to have higher levels of bioactivity than existing whole mussel composition products and consequently they are likely to have increased health benefits. No anti-oxidants, surfactants, co-surfactants, emulsifying agents or other additives are required to be added to the compositions in order to maintain the bioactivity or to otherwise stabilise the compositions. The compositions are therefore completely natural.

One of the key advantages of the compositions of the invention is that due to the unique emulsion-like structures the bioactive components are more easily or readily absorbed into the body (via skin, tissues, cells and/or bloodstream), therefore the compositions of the invention will be an effective means of delivering beneficial bioactive components as the structures and properties of the compositions allow for improved bioavailability. Without being bound by theory, the inventor believes that the compositions are self-emulsifying so the bioactive components present in the compositions of the invention will be easily absorbed into the body via paracellular absorption between cells. Literature suggests that self-emulsifying formulations, when given orally, may offer improvements in both the rate and extent of absorption of the bioactive compounds present in the composition and also the consistency of the resulting plasma concentration profiles. The bioactive components of the compositions of the invention will therefore be delivered effectively. Furthermore, the property of self-emulsification permits the compositions of the invention to be administered in concentrated form, as for example in an encapsulated format, with the expectation that a fine emulsion will be formed in any targeted location in the digestive tract.

Without wishing to be bound by theory, it is believed that the compositions of the invention comprise physiochemical components which naturally function as surfactants and/or co-surfactants and/or emulsifiers and thereby act as natural solubility enhancers in a self-emulsifying system. Therefore, the lipid or lipophilic bioactive components present in the hydrophobic phase of the composition are dispersed in a stable and homogenous manner through the continuous or hydrophilic or aqueous phase. It has been found that at least some of the particles in the hydrophobic phase have a layer surrounding or encapsulating the particles or droplets or globules wherein one or more lipid or lipophilic bioactive components are located inside the globules and are protected by the surrounding layer. It is possible that these particles or globules are lipoproteins or similar. The continuous or hydrophilic phase of the composition has one or more bioactive components dispersed or suspended therein, and may also comprise some solid particles in solution or suspension.

It is likely that the components that function as surfactants and/or co-surfactants and/or emulsifiers comprise low molecular weight proteins and/or peptides as these appear to remain on the surface of the particles or globules that are dispersed through the hydrophilic phase. These substances could assist in forming the structured particles or globules which then repel each other and the repulsive forces cause them to remain stably suspended in the hydrophilic phase. Alternatively or additionally it may be that the substances modify the viscosity of the composition which could help to create and maintain the suspension of the hydrophobic particles or globules in the hydrophilic phase.

Description of Best Method

The compositions or extracts of the invention can be produced in a number of different ways, but are preferably produced using enzyme hydrolysis methods. It has been found that improved compositions of the invention can be made by enzyme hydrolysis of fresh or live undamaged or unprocessed mussel material, that is, mussel material that has not been processed or comminuted before any application of enzymes. One of the best methods of preparing a composition of the invention known to the inventor at the time of filing this application will now be described.

As shown in FIG. 1, a liquid composition of the invention can be produced from whole fresh (preferably live) mussel starting material. The method comprises applying at least one enzyme formulation comprising one or more enzymes to at least a portion of the mussels and leaving the mussels in contact with the enzyme formulation for a sufficient period of time to separate the target substrate(s) and/or target biological material from the shells of the mussels and substantially liquefying the target biological material and/or target substrate(s). The enzyme formulation firstly separates the meat or flesh and other biological material from the shells of the mussels and secondly gently and progressively breaks down the biological material into smaller particles, thereby releasing bioactive components including nutrients, functional molecules and bioactive compounds. Any residual non-target materials or substrates, namely solids such as shells and/or fragments thereof can then be removed from the resulting liquid composition. Preferably the method includes a warming step to serve the dual purpose of opening or gapping the mussel shells (if they are alive) and activating the enzyme formulation to speed up the process. The liquid composition can then be dried to produce a dried composition.

The whole fresh (preferably live) mussels are preferably cleaned and processed as soon as possible after harvesting, and processed fresh and preferably alive, or at least within 12 hours, and preferably within three hours post-mortem. If it is not possible to process the shellfish quickly after harvesting, the mussels can be cleaned and stored in cold storage (at about 4-9° C., ideally 7° C.) for up to 48 hours before processing. Cold storage may be preferred in some cases because the sea water drains out naturally which is helpful to reduce water content for later drying of the composition.

At least one enzyme treatment step (10) is carried out which is designed to remove or separate target biological material, for example the meat and/or other biological material present in or on the shells of the mussels, from the shells of the mussels, and at the same time gently liquefy or reduce the size of the target biological material to produce a liquid emulsion-like composition (11). The enzyme treatment step involves the exposure of one or more target biological materials or target substrates of the mussels to an enzyme formulation, comprising one or more enzymes that are suitable for acting on the target substrate(s).

If whole mussels are used, it is necessary to open or gap the mussels, or pierce or penetrate at least a portion of the shells in some manner in order to expose at least a portion of the interior of the shells containing the meat and other biological material to the enzyme formulation. This is preferably done by application of gentle heat as noted above, or it can be done by an HPP process, or other processes such as laser opening methods localised to the abductor muscle to trigger gapping or opening. If other piercing or cracking methods are used, these are preferably gentle methods which cause minimal disturbance to the biological material inside the shells. If whole live mussels are used, the mussels are preferably opened or gapped by a warming step, and preferably the mussels are alive at or up to the point of the gapping or opening step. An enzyme formulation comprising one or more enzymes selected to act on the abductor muscles as the target substrate can be used to facilitate the full opening of the bivalves once they are gapped or partially opened by the warming step.

The warming step can be carried out by any means known in the art, for example, by application of a heat source directly or indirectly to the shellfish. Preferably the warming step is carried out by way of application of steam (e.g. flash steam injection or infusion) at a temperature of about 90-100° C. to quickly achieve a temperature of about 35-55° C. in or around the shellfish. Alternatively, the warming step could be carried out by the use of a thermal jacket or other thermal means to heat the shellfish to the optimum temperature, however this would be a slower process.

The or each enzyme formulation can comprise one or more types of enzymes sourced from animal, plant or microbial origins, or a combination of one or more enzymes with one or more acids or alkalis. The selection of enzymes requires consideration of the species of mussel being processed, the target substrates to be acted on, the form of composition desired, the processing equipment used and the factors that will influence and/or facilitate enzyme activity.

Examples of enzymes that may be used in the enzyme formulation include but are not limited to the following: lipase, phospholipase, phosphatase, glycogen phosphorylase, glucosyltransferase, glucosidase, proteinase, collagenase, glycogen debranching enzymes, phosphoglucomutase, cellulases, chitinases, polysccharidases, disaccharidases, alginase, amylase, maltase, peptidase, pepsin, thrombin, trypsin, α-Amylase (from malted cereals), β-Amylase (from sweet potato or malted cereals), actinidin (from kiwifruit), ficin (from figs), bromelain (from pineapple), papain (from papaya), and enzymes derived from the following microorganisms: Bacillus amyloliquefaciens, Bacillus subtilis, Bacillus licheniformis, Bacillus coagulans, Bacillus acidopullulyticus, Bacillus halodurans, Aspergillus melleus, Aspergillus oryzae, Aspergillus niger, Lactococcus lactis, Geobacillus stearothermophilus, Rhizomucor miehei, Micrococcus luteus, Penicillium funiculosum, Trichoderma reesei, Trichoderma viride, Escherichia coli, Kluyveromyces lactis, Paenibacillus macerans, Chaetomium gracile, Penicillium lilacinum, Saccharomyces cerevisiae, Bacillus circulars, Kluyveromyces marxianus, Trichoderma harzianum, Disporotrichum dimorphosporum, Humicola insolens, Talaromyces emersonii, Rhizopus delemar, Rhizopus oryzae, Rhizopus niveus, Hansenula polymorpha, Penicillium camembertii, Candida rugosa, Mucor javanicus, Penicillium roquefortii, Rhizopus arrhizus, Cryphonectria parasitica, Streptomyces violaceoruber, Klebsiella pneumoniae, Streptomyces mobaraensis, Lactobacillus fermentum, Actinoplanes missouriensis, Microbacterium arborescens, Streptomyces olivaceus, Streptomyces olivochromogenes, Streptomyces murinus, Streptomyces rubiginosus and Clostridium histolyticum.

The enzyme formulation may further include one or more acids or alkalis such as phosphoric acid, sulphuric acid, tannic acid, citric acid, tartaric acid, sodium hydroxide, ammonium hydroxide, magnesium hydroxide, potassium hydroxide.

The or each enzyme formulation can be pre-mixed and applied to the mussels, or each component of the or each enzyme formulation can be applied to the mussels separately either at the same time or sequentially during the enzyme treatment step(s).

The amount of the or each enzyme formulation used depends on the type of mussels being processed, as well as the operating parameters (e.g. temperature, pH, time and end point) set by the user, and desired product specifications. The amount of each enzyme included in the or each enzyme formulation used should be calculated based on the amount of the or each target substrate to be acted on which can be estimated based on the weight of the whole fresh shellfish raw material. For example, in a 10 kg batch of mussels, there will be approximately 5 kg of flesh or meat and water (with the remaining 5 kg being shells). There is approximately 12% protein in 5 kg of mussels so in order to effectively liquefy the protein component or the protein-based substrates, the amount of the or each proteolytic enzyme included in the or each enzyme formulation would be calculated based on the estimated 12% protein substrate, not based on the mass of the starting shellfish material. Preferably the amount of the or each enzyme included in the or each enzyme formulation is in the range of 0.1-10% calculated based on the estimated amount of the or each target substrate to be treated in the or each enzyme treatment step.

Many commercially available enzymes have been tested and are effective. The selection of enzymes is generally a balance between cost and the overall efficiency of the enzyme formulation at the particular operating parameters used, and taking into account the desired end-product specifications.

Enzymes and/or combinations of enzymes that have been found to be particularly effective in the processing of green-lipped mussels, include one or more enzymes selected from the group comprising enzymes derived from bacterial strains that produce subtilisin, including Bacillus amyloliquefaciens; enzymes derived from Bacillus licheniformis, Bacillus subtilis, Aspergillus niger and Aspergillus oryzae; cysteine proteases; carbohydrases, sucrase, amylase, lipase, phospholipase, phosphatase, esterases, and catalase.

In one preferred method the enzyme formulation comprises a combination of at least two enzymes selected from the group comprising enzymes derived from Bacillus amyloliquefaciens, enzymes derived from Bacillus licheniformis, cysteine proteases, and enzymes derived from Aspergillus oryzae, wherein at least one of the enzymes is a proteolytic enzyme.

The optimum pH for processing mussels is in the range of pH 2-9, preferably about pH 4 to 8, although some enzymes may work at a lower pH. The pH can be adjusted as and when necessary during the process by the addition of a suitable acid or alkali.

The one or more enzyme treatment steps are preferably carried out under temperature controlled conditions and for a specified time period. The reaction temperature is preferably no more than 60° C., and is preferably in the range of about 20-60° C. The total reaction time is preferably less than 120 minutes, more preferably less than 90 minutes and more preferably is in the range of 15-40 minutes. The reaction temperature and duration should be calculated based on the desired end-product specifications. The reaction time is generally set based on the minimum time required to achieve the desired end-product specifications.

It has been found that duration of between about 15-40 minutes for each enzyme treatment step is sufficient for achieving a significant degree of hydrolysis.

It has been found that improved compositions can be obtained if the enzyme hydrolysis process is very short and the starting material is as fresh as possible and preferably live.

An agitation step is preferably used during and/or after the warming step and/or the enzyme treatment step, which enables the shellfish to be continually moved and therefore more evenly exposed to the increased temperature and/or the enzyme formulation. After the enzyme treatment step(s) (10), the resulting composition is in the form of a liquid composition (typically of slurry like consistency) that resembles an emulsion or a colloid (11). The liquid composition is then subjected to at least one separation step (12) to remove any shells, shell fragments, or other large non-target solid biological material from the composition. The separation step can be carried out by any means known in the art, for example, by the use of screens, filters or sieves, or a combination thereof. A series of separation steps may be carried out to obtain a liquid composition with a desired particle size or particular emulsion structure or for better recovery of certain bioactive components. Preferably this liquid composition is then siphoned or drained off or otherwise recovered, and the remaining material is recycled back into the process and retreated with one or more enzyme formulations comprising the same or different enzymes in one or more subsequent enzyme treatment steps (14) so that the larger particles are further reduced, broken down or converted into smaller particles until the desired particle size or particular emulsion structure or certain level of bioactive components are achieved in respect of substantially all of the mussel starting material other than the shells. This means that there is minimal waste and minimal yield loss.

In one preferred method, the main steps of the process are carried out in a single treatment vessel or chamber, as shown in FIG. 2. The treatment chamber (20) is preferably a cylindrical shaped vessel which can be sealed and pressurised. For batch processing, any amount (e.g. kilograms or tonnes) of whole fresh shellfish may be placed or conveyed from a hopper or other storage container into the treatment chamber (20). The treatment chamber may have a built-in weigh cell so that the size of each batch can be controlled and monitored. This may not be necessary for continuous or semi-continuous processing methods. The amount of shellfish processed at any one time in the treatment chamber will depend on the internal size and volume of the treatment chamber. There will need to be some void space within the treatment chamber after the shellfish has been added to hold the heat/steam and to give space for movement.

Preferably the treatment chamber is orientated horizontally, rather than vertically, or it is orientated in a sloped position. This avoids the need for a mechanical crushing step, and also avoids the need to add water to the treatment chamber for processing. If a steam injection is used as a heating means, then it is possible to process the mussels in this way without adding any other water to the treatment chamber.

The treatment chamber (20) may include at least the following features and components: a sealable opening (21); a heating means (22); a dosing system for the enzyme formulation (23); and an agitating means (24).

The sealable opening (21) can be used both for introduction of the shellfish into the treatment chamber, and for discharging the resulting liquid composition after the enzyme treatment step(s).

The heating means (22) may be a steam heating device such as a flash steam injector or infuser located inside the treatment chamber. Preferably the steam injector or infuser is located in a position within the chamber to enable the steam to be delivered into the central part of the chamber. In another option, the heating means can include a heating element or thermal jacket or heat exchanger (25) located on or near at least one of the walls of the treatment chamber, in place of or in combination with the steam injector or infuser. The heating means is preferably operated to raise the internal temperature of the treatment chamber to between about 35-60° C. in order to condition the shellfish for the enzyme treatment step, by bringing the treatment chamber to an optimum temperature to activate the enzyme formulation, and in the case of whole live mussels to partially open or gap the mussels so that the enzyme formulation can be distributed inside the shells. If a flash steam injection or infusion is used as the heating means, then preferably steam is injected at a temperature of about 90-100° C. for a predetermined time period (generally very short, for example between about 90-120 seconds) in order to quickly but gently raise the internal temperature of the treatment chamber to the optimum temperature. The steam injection or infusion time is dependent on infeed raw material temperature, the size of the treatment chamber, the type of equipment (e.g. whether another heating source such as a heating jacket is also used and if so, whether this is on or off), the efficiency of agitation, the nozzle size of the steam injector or infuser and the volume of steam injected.

The dosing system (23) may include an automatic dispensing device to which the enzyme formulation(s) can be added, which is connected to a dosing means (26) located inside the treatment chamber, so that dosing can be controlled. Preferably the enzyme formulation is poured or sprayed onto the shellfish by way of the dosing means (26) which may have or comprise for example a spray nozzle to facilitate distribution of the enzyme formulation onto the shellfish. Preferably the dosing means (26) is positioned in such a manner to enable substantially even distribution of the enzyme formulation onto the shellfish. The enzyme formulation can be added either before, at the same time, or after the warming step is commenced.

Preferably the treatment chamber (20) or the contents of the treatment chamber are able to be continuously or semi-continuously rotated or agitated. This provides for more effective distribution of heat in the treatment chamber, and for more effective distribution of the enzyme formulation to the contents of the treatment chamber.

The treatment chamber may also include a recycling system (27) whereby the contents of the vessel can be re-circulated. For example, the enzyme formulation could be re-circulated and re-used, or the liquid shellfish material can be re-circulated during the enzyme treatment step(s) or recycled for subsequent enzyme treatment steps (using enzyme formulations comprising the same or different enzymes) to be carried out after some of the liquid composition is siphoned off or removed. The recycling system may be a pipe or circulation tube extending from an outlet (29) at or near the base of the treatment chamber and providing a fluid pathway back to the dosing means (26) or other suitable inlet port which may be located at or near the top of the treatment chamber.

The internal temperature of the treatment chamber may be monitored by an external temperature gauge or the like (connected to an internal temperature probe) so that the temperature is maintained at the ideal processing temperature (less than 60° C., preferably between 55-60° C.) for the duration of the enzyme treatment step(s). The temperature is maintained by either the heating source (25) being set at the desired temperature for the duration of the reaction time, or by applying further direct flash steam through the steam injector or infuser as and when necessary to maintain the optimum internal temperature. The duration of the first enzyme treatment step is determined by the amount of time it will take to substantially remove or separate the target biological material and/or target substrate(s) from the shells of the mussels, and substantially liquefy the target biological material and/or target substrate(s) with the selected enzyme formulation. Generally, the enzyme treatment step/liquefying process will take less than 120 minutes in total and more preferably less than 90 minutes in total, however in order to achieve complete release or break down of some bioactive components, a longer time period may be required. Preferably though, the duration of the or each enzyme treatment step is between about 15-40 minutes. The treatment chamber may comprise an exhaust system (28) which is activated at the conclusion of the enzyme treatment step, that is, the treatment chamber is stopped or deactivated and the exhaust is opened to expel the heat or steam and pressure within the treatment chamber.

After the first enzyme treatment step is completed, the target biological material and/or target substrates of the shellfish starting material will have been reduced to a predominantly liquid composition, in the form of an emulsion-like composition or colloid (typically of a slurry like consistency), comprising some solid material such as shells and shell fragments, and other solid biological material comprising non-target substrates, for example, byssus threads (if undesired). The liquid composition is discharged from the treatment chamber (via the sealable opening or other discharge port) and is subjected to at least one separation step (12). The first separation step is carried out to remove residual shells and/or shell fragments, and any other large solid non-target material from the liquid composition. The clean shells may be collected in a sieve or other filtration device and discarded, or removed by conveyor into a container. The residual shells and other solid waste material (for example byssus threads if undesired) may be further processed into other commercial products if desired (for example liquid or dried products for use as pet foods or in animal feeds, or beard/shell products for industrial uses). Alternatively, some of the separated solid material may be retreated with one or more different enzyme formulations in one or more subsequent enzyme treatment steps if certain bioactive components are desired to be released from this material.

After the separation step, a series of filtration steps (13) may be conducted to progressively reduce the particle size of the liquid composition, by means known in the art, for example, by the use of one or more screens, filters or sieves, with openings of progressively decreasing diameter. Preferably at least one filtration step is carried out after the separation step in order to remove large particles from the liquid composition after the residual shells have been removed. Preferably the liquid composition is able to be filtered to a particle size of less than 200 μm in one filtration step after the separation step. A particle size of less than 200 μm is especially important if spray drying is to be used to dry the composition.

The material remaining (that is, the non-target biological material comprising non-target substrates) after the separation step and/or after the first filtration step (including any remaining active enzyme formulation) may be added back into the treatment chamber (by a recycling system or otherwise) and subjected to one or more further enzyme treatment steps (typically using a different enzyme formulation) to progressively liquefy and emulsify other substrates in the remaining material, in order to release further bioactive components.

The liquid composition may be stabilised (15) before or after the separation or filtration step(s) (if carried out), in order to deactivate the enzyme(s) and to pasteurise or sterilise the liquid composition, to meet food safety requirements. Deactivation of the enzyme(s) can be achieved by a number of means known in the art, for example by application of flash heat treatment (such as UHT, HTST, PEF), or by altering the pH of the liquid composition to a pH at which the enzyme(s) become deactivated (i.e. pH<4 or pH>10), for example, by addition of tartaric acid or other acids. Because pH stabilisers could adversely affect some of the bioactive components in the liquid composition or result in separation/denaturation of some components, the preferred stabilisation method is rapid heat treatment. For example, a heat exchanger may be used to quickly increase the temperature of the composition to above 80° C. for a short time period (for example up to 85° C. for 5-15 minutes). Alternatively, a further steam injection or infusion (controlled by a temperature probe) could be applied at the end of the enzyme treatment step to increase the internal temperature of the treatment chamber to this level before the exhaust is activated. Other methods of stabilisation not involving heat treatment may be used such as microfiltration or ultrafiltration methods.

After stabilisation and filtration, the liquid composition can be used as is, or it can be dispensed into containers and stored at low temperature for later use, or it can be immediately frozen for later use. If the liquid composition is frozen immediately the stabilisation step may not be required, however the composition will most likely need to be stabilised in some manner once it is thawed for later use.

In FIG. 1, a drying step (16) is carried out after the separation step (12) in order to produce a dried composition. The liquid composition can be dried using any known drying methods in the art. Preferably drying is carried out by low temperature (<80° C.) drying means such as freeze drying, or by flash drying means such as spray drying, vacuum drying or belt drying. After drying, the dried composition may be ground or milled into a powder (17) by methods known in the art. The liquid or dried compositions can be further processed by one or more separation, fractionation or extraction steps to produce other product formats (18) as discussed further below.

FIG. 3 provides a more detailed schematic representation of the best method including the use of a treatment chamber as shown in FIG. 2 and including subsequent possible processing steps.

A specific advantage of the liquid compositions of the invention is that they comprise a high percentage of material of small particle sizes in comparison to existing whole mussel composition products. For example, in one study, the inventor found that a green-lipped mussel composition of the invention had about 60% of 40-50 μm sized particles, in comparison to a prior art whole mussel composition which had a majority of particles sizes in the range of 300-1200 μm. There is also a much higher concentration of particles or globules in the aqueous phase of the compositions of the invention compared to prior art products. Studies have shown that the majority of particles in the compositions of the invention are micro-particles with sizes in the range of about 100-50,000 nm.

FIG. 11 shows images obtained from an FEI Nova NanoSEM 450, high resolution, field emission gun scanning electron microscope (FEG-SEM), of the microstructure of a dried green-lipped mussel composition of the invention in comparison to an existing product produced by mechanical processing methods. The images show a clear distinction in particle shape and size. The compositions of the invention have much smaller spherically shaped particles and more uniform particle structure and size. FIG. 12 shows typical images of the emulsion-like structures of dried green-lipped mussel compositions of the invention reconstituted with water. The rehydrated compositions are stable in aqueous suspension and form unique microstructures comprising structured spherical biomaterials comprising both lipids and proteins, which could be lipoproteins or particles comprising lipid bilayers or similar. The images show that the compositions comprise a mixture of material of varying size including some nano-particles or nano-droplets and some micro-particles or micro-droplets.

It is believed that lipids and/or lipophilic bioactive compounds are encapsulated and protected inside the structured particles in the hydrophobic phase, and hydrophilic and possibly other bioactive compounds are dispersed or suspended in the hydrophilic phase.

Due to the small and uniform particle size of the compositions of the invention, various drying methods are possible, including spray drying.

The small particle size and stable and uniform nature of the liquid compositions of the invention also allow for other direct non-thermal sterilization processes, such as Pulsed Electric Field (PEF) or Ultra-High Temperature (UHT), or High Temperature/Short Time (HTST) pasteurization to be used as necessary to suit any possible applications.

A further advantage of the compositions of the invention is that they comprise more amino acids and small proteins and/or peptides, some of which are essential amino acids, some of which are flavour enhancers and some of which are functional amino acids and peptides. The inventor has found that the compositions of the invention have improved sensory attributes including smell, taste or flavour profiles due to the increased amount of flavour enhancing amino acids. See FIGS. 9 and 10. FIG. 9 shows that dried green-lipped mussel compositions of the invention have a less fishy and grassy flavour profile than existing products, and that they have a sweeter flavour profile. FIG. 10 shows that dried green-lipped mussel compositions of the invention have lower levels of sodium and chloride on the particle surfaces and are therefore less salty than existing products.

The compositions of the invention have an increased yield of bioactive components and an unexpected and highly desirable microstructure which is expected to increase the bioavailability of the bioactive components. Advantageously, if the compositions are made by the enzyme hydrolysis method described above, no antioxidants are required to be added during or after processing in order to maintain the bioactivity of the compositions. Furthermore, the compositions are stable and no surfactants, co-surfactants or emulsifiers are required to be added in order to maintain the stability of the compositions.

The liquid or dried compositions of the invention can either be used as is, or be formulated into other finished products in various dosage formats including oral dosage formats, topical dosage formats and other dosage forms for various uses as described below.

Further Processing of Liquid and Dried Compositions

The liquid and dried compositions of the invention can be used as is or formulated for use for a wide variety of purposes including as foods, food supplements, food ingredients for use in food applications, or for cosmetic, pharmaceutical, nutraceutical or veterinary applications. Alternatively, the liquid and dried compositions of the invention can be used or sold as intermediate products intended for further processing into any number of different extracts and/or product formats including dietary supplements, nutraceutical compositions, veterinary compositions, pharmaceutical compositions or cosmetics. The uniform particle size and stable nature of the compositions make them desirable for further processing to obtain extracts and other product formats comprising high levels of bioactive components with improved bioavailability.

Possible dosage forms include without limitation, oral dosage forms such as tablets, capsules, dried powder formats, oils, food ingredients; topical dosage forms for external use such as creams, gels, emollients, ointments, lotions, dressings such as plasters, bandages and medicated dressings; and other internal dosage forms including injectable forms.

Both the liquid and dried compositions of the invention can be subjected to one or more fractionation, separation or extraction steps by methods known in the art to yield different useful products. For example, the compositions can be further separated into various fractions, including but not limited to a hydrophobic or lipid-rich fraction, and a hydrophilic fraction containing water soluble proteins, peptides, amino acids, nucleic acids, minerals, carbohydrates, vitamins, biotin and others, and water insoluble (high molecular weight materials) and undissolved proteins etc. It has been found that due to their structure the dried compositions of the invention have very good extractability characteristics and can produce high quantitative yields of extracted components with high bioactivity.

The lipid rich and/or hydrophilic extracts can be formulated into various formats including food flavourings, food ingredients, oils, pharmaceutical compositions or nutritional supplements such as tablets, capsules, cachets, syrups, elixirs, oils or other dosage forms using suitable carriers and excipients. Due to the increased yield of bioactive components in the liquid and dried compositions of the invention, it is putated that any extracts produced therefrom will have increased concentrations of bioactive components and improved bioavailability.

Examples of Possible Product Formats

It is envisaged that the following product formats (non-limiting) could be derived from further processing of either the liquid or dried mussel compositions of the invention:
Oils—in liquid form (including encapsulated form in either hard-shell capsule form or soft gel form), dry form including tablets or powders, or oil with carrier form, for use in dietary supplements, pharmaceutical or nutraceutical products, cosmetics or veterinary products;
Liquids (either whole mussel compositions (including the lipid rich fraction), or fractionated mussel liquids (with the lipid rich fraction removed) for use in dietary supplements, pharmaceutical or nutraceutical products, cosmetics or veterinary products;
Powders (either whole mussel powders (including the lipid rich fraction), or fractionated mussel powders (with the lipid rich fraction removed) for use in dietary supplements, pharmaceutical or nutraceutical products, cosmetics or veterinary products;
Food ingredients such as food flavourings, seasonings, ready made sauces, meals etc.
Pet foods

### EXAMPLE 1

Green-lipped mussels (Perna canaliculus) were processed by adding 60 kg of live whole mussels to a sealable, pressurisable treatment chamber. The chamber was closed a flash steam injection was applied at a temperature of 100° C. for a period of 90 seconds. At the same time the chamber was rotated for about 5 minutes to achieve an optimum temperature of about 45-50° C. evenly distributed inside the chamber in order to open or gap the mussels and to condition the mussels for the enzyme treatment step. The chamber was then opened and 6% of an enzyme formulation (based on the total protein amount of between 3-4 kg) was manually added (in liquid form). The enzyme formulation comprised a protease enzyme derived from Bacillus species (Bacillus licheniformis commercially available as ESP153). The internal temperature of the treatment chamber was maintained at about 55-60° C. by the initial steam injection (no further steam was required). The chamber was rotated for a period of about 40 minutes. At the end of this time period the chamber was deactivated by activation of the exhaust which expelled the heat and pressure from the chamber. The contents of the chamber were then discharged onto a separating screen to remove any residual shells, shell fragments and any other large particles. The liquid composition was then filtered through a 200 μm mesh filter.

FIG. 4 shows a microscopy image and associated graph of the particle size distribution of the resulting liquid composition. The particle sizes range from 1 μm to 100 μm, with the majority of particle sizes being between 1 μm-50 μm. There is a high proportion of particles between 1 μm-10 μm. The liquid composition comprises an emulsion-like composition of suspended particles (in an aqueous medium) of uniform size and distribution. The emulsion could be a double or multiple emulsion comprising low molecular weight proteins/peptides and other substances which appear to remain on the surface of the oil/water droplets and assist in stabilising the composition.

After the separation and filtration steps, the liquid composition was dried by freeze drying, without any stabilisation step. FIG. 5 is a graph showing the particle size distribution of the dried green-lipped mussel composition produced in this example of the invention. The dried composition is rehydrated. The particle sizes range from 1 μm to 100 μm, with the majority of particle sizes being between 10 μm-50 μm.

About 45-50% yield of liquid composition was obtained in this example (i.e. about 25-30 L of liquid composition). From that, about 6-7% yield of dried composition was obtained (i.e. about 3-4 kg). The dried composition had a moisture content of less than 6%. The dried composition was highly soluble and could be readily rehydrated in aqueous solution to achieve a stable composition substantially the same as the original liquid composition (as shown in FIG. 4).

FIG. 6 provides an analysis of the main components of the dried green-lipped mussel composition produced in this example. As shown in FIG. 6, the components of the composition are different to that of a dried composition produced by a typical mechanical process (in the comparative conventional process live mussels were manually opened, the meat or flesh removed and minced and then freeze dried). The dried composition produced in this example comprises about 10% lipids, 32% proteins and 29% other soluble components in its aqueous phase, and about 2% lipids, 18% proteins and 9% other non-soluble components. In comparison, the dried composition produced by conventional mechanical processing comprises about 3% lipids, 5% proteins and 29% other soluble components in its aqueous phase, and about 8% lipids, 45% proteins and 10% non-soluble components.

Given that the composition of biological material present in mussels varies between seasons, the dried composition of the invention is likely to comprise about 7-16% lipids and about 45-55% protein. Advantageously, it is expected that a dried composition of the invention would comprise >85% of soluble proteins and other components in its aqueous phase. In comparison, the dried composition produced by conventional mechanical processing comprises typically only 25% of soluble proteins and other components in its aqueous phase.

There is over 70% hydrophilic fraction in the composition of the invention which is almost double the 37% hydrophilic fraction in the composition produced by conventional mechanical processing methods. The compositions of the invention have more bioactive components in a highly bioavailable form. It is envisaged that the non-soluble portion could be further processed using one or more different enzyme hydrolysis steps to break down or convert the non-soluble material to release further bioactive and soluble components.

Bioactivity Studies

The dried composition of Example 1, together with one other dried composition that was prepared in the same way but dried by spray drying rather than freeze drying, were tested for their anti-inflammatory properties, in comparison with two existing dried mussel extracts.

The relative anti-inflammatory properties of the test samples were determined by establishing their abilities to inhibit the activation of neutrophils as measured by the production of superoxide. The efficacy of the test samples was referenced against Aspirin, as well as an unsupplemented control group.

Details of the test samples are set out in the following table:

Description

Competitor A (Whole dried green lipped mussel extract prepared by
mechanical crushing and homogenising followed by drying)
Competitor B (Whole dried green lipped mussel extract prepared by
mechanical crushing, centrifuging followed by freeze drying)
Sample 1 (Whole dried green lipped mussel extract prepared as per
example 1 (with freeze drying))
Sample 2 (Whole dried green lipped mussel extract prepared as per
example 1 (with spray drying))

Samples 1 and 2 were produced from the same batch of mussels. Each of the above test samples was extracted with ethanol at a ratio of 1:10 (w:v) and the residues were then extracted with distilled water at the same ratio, so that the activity of both the lipid rich or hydrophobic fraction and the hydrophilic or aqueous fraction of each of the test samples could be tested. The experimental procedure for determining the effects of the test samples on inflammation was based on the methods described in Tan, AS and Berridge, MV (2000). Superoxide produced by activated neutrophils efficiently reduces the tetrazolium salt, WST-1 to produce a soluble formazan: a simple colorimetric assay for measuring respiratory burst activation and for screening of anti-inflammatory agents. J Immunol. Meth. 238: 59-68. Neutrophils were harvested from rat whole blood and activated with phorbol 12-myristate 13-acetate (PMA). The activated neutrophils were then incubated and cultured in the presence of each of the test samples and the controls. The reduction of the WST-1 dye was measured to determine the products of superoxide. The control group was set at 100% activity (0% inhibition) and the inhibition of all samples were compared to this reference. Aspirin is a known anti-inflammatory compound and so was tested for a reference and exhibited 50.2% inhibition at a concentration of 400 μg/ml (16.5% inhibition was exhibited at a concentration of 100 μg/ml, and 48.12% inhibition was exhibited at a concentration of 200 μg/ml, showing a dose response effect).

The ethanol and water extracts (lipid rich and hydrophilic fractions) from each of the test samples were tested at 400 μg/ml for their anti-inflammatory activity based on the inhibition of superoxide by activated neutrophils. The yields of each extraction were used along with the activity of each to obtain an estimate for the total activity within each of the test samples for the two fractions. The results are shown in the tables below.

Ethanol Extracts—the Relative Contribution of the Lipid Fraction in each of the Samples Tested for Anti-Inflammatory Activity

| | | | Weight (mg) of |
|---|---|---|---|
| | | | powder required to |
| | | | achieve 100% |
| | % of total | % Inhibition | inhibition of |
| Sample | weight | (400 μg/ml) | inflammation |
| Competitor A | 6.22 | 81.01 | 7.9 |
| Competitor B | 6.13 | 100 | Cannot be |
| | | | determined |
| Sample 1 | 8.58 | 98.86 | 4.71 |
| Sample 2 | 10.05 | 100 | Cannot be |
| | | | determined |

Water Extracts—the Relative Contribution of the Hydrophilic Fraction in each of the Samples Tested for Anti-Inflammatory Activity

| | | | Weight (mg) of |
|---|---|---|---|
| | | | powder required to |
| | | | achieve 100% |
| | % of Total | % Inhibition | inhibition of |
| Sample | Weight | (400 μg/ml) | inflammation |
| Competitor A | 55.09 | 26.26 | 2.96 |
| Competitor B | 41.37 | 30.09 | 3.43 |
| Sample 1 | 85.02 | 28.6 | 1.8 |
| Sample 2 | 81.21 | 35.47 | 1.56 |

The results show that all of the samples showed some degree of anti-inflammatory activity, with the activity in the lipid fractions being higher than the activity in the hydrophilic fractions. It was however surprisingly discovered that there is anti-inflammatory activity in the hydrophilic fractions, so both the lipid and hydrophilic fractions of the mussel extracts contribute to the overall anti-inflammatory activity of the test samples. FIG. 7 is a graph showing the results of this study in terms of the total bioactivity of the test samples (i.e. lipid and hydrophilic fractions combined) based on IC50 of aspirin equivalent (at 400 μg/ml). This graph shows that the test samples of example 1 show a much higher level of bioactivity than existing whole green-lipped mussel composition products. This is likely due to the combination of the comparatively high yields of the hydrophilic fractions of the test samples produced by example 1, with the good yields and high activity of the lipid fractions which gives an overall increased bioactivity of the extracts as a whole. Very small dosages of the hydrophilic fraction of the test samples produced in example 1 are required in order to achieve 100% inhibition. Similarly, very small dosages of the extract as a whole would be required in order to achieve 100% inhibition. The results show that there is an increased yield of bioactive components in the compositions of the invention.

The results of this study are further supported by a subsequent study which was carried out on the same test samples to assess the DPPH scavenging activity of each of the test samples.

The antioxidant activity of each of the samples was tested using the DPPH scavenging method (i.e. by using the stable free radical 2,2-Diphenyl-1-(2,4,6-trinitrophenyl) hydrazyl as a substrate). Whilst the DPPH method is not a direct anti-inflammatory assay, antioxidant activity has been an indication of anti-inflammatory activity in many study cases. The DPPH solution was prepared in 0.1 mM ethanol and kept in the freezer in the dark before use. The positive control was ascorbic acid prepared as 0.1 mg/ml in a buffer containing citric acid and NaHPO₄ (pH 5). Equal amounts of sample solution and DPPH solution were added together, and the assay tube or plate was incubated for 30 minutes in the dark, followed by absorbance measurement at 517 nm on a spectrophotometer. In the blank control experiment of each sample, DPPH was replaced with ethanol. In the DPPH blank control experiment, the sample was replaced with the media (water or solvent) the sample was prepared in.

The scavenging activity (DPPH inhibition%) is calculated by percentage of the absorbance from the sample versus the DPPH only:

$$DPPH inhibition \% = [ 1 - \frac{( A sample - {A}_{sample blank} )}{( {A}_{DPPH} - {A}_{DPPH blank} )} ] \times 100$$

All samples were tested at a concentration of 10 mg/ml. The results showed that all samples had antioxidant activity (above 80% inhibition in all samples). The results are summarised in the tables below:

The IC₅₀ values of DPPH inhibition in water extracted samples

| Sample | A | B | 1 | 2 |
|---|---|---|---|---|
| IC₅₀ | 6.90 | 2.96 | 3.01 | 3.02 |
| (mg/ml) | | | | |

The IC₅₀ values of DPPH inhibition in ethanol extracted samples

| Sample | A | B | 1 | 2 |
|---|---|---|---|---|
| IC₅₀ | 8.12 | 6.34 | 4.05 | 4.50 |
| (mg/ml) | | | | |

These results are shown in FIG. 8 which is a graph showing the results of this study in terms of the total bioactivity of the test samples (i.e. lipid and aqueous or hydrophilic fractions combined) based on IC50 of Vitamin E equivalent (at 9.26 μg/ml). It is clear from the results that the samples of example 1 exhibit stronger antioxidant activity than existing whole green-lipped mussel composition products. Both the water extracts and the ethanol extracts contribute to the overall antioxidant activity.

### EXAMPLE 2

Two liquid mussel compositions were prepared by subjecting whole fresh mussel meat (uncomminuted) to an enzyme treatment step. The first composition was prepared by applying 1% of an enzyme foiiiiulation comprising ESP153 to the mussel meat at a temperature of 55° C. for a period of 60 minutes with gentle agitation. The second composition was prepared by applying 2% of an enzyme formulation comprising ESP153 to the mussel meat at a temperature of 55° C. for a period of 60 minutes with gentle agitation.

The resulting liquid compositions were tested for anti-inflammatory activity using a Cyclooxygenase (COX, also called prostaglandin H synthase or PGHS) assay. Cyclooxygenase is a bifunctional enzyme exhibiting both COX and peroxidase activity. Recent research has established that there are two distinct isoforms of COX: COX-1 and COX-2. COX-1 is expressed in a variety of cell types and involved in normal cell biology. COX-2 is induced by mitogenic stimuli (LPS and cytokines) and is responsible for the biosynthesis of prostaglandins (PGs) under acute inflammatory conditions and therefore it is a target enzyme for the anti-inflammatory activity of nonsteroidal anti-inflammatory compounds. An ideal anti-inflammatory candidate should only possess COX-2 inhibition, not COX-1 inhibition. COX-2 colorimetric inhibitor screening assay kits from Cayman Chemical Company (MI, USA) were used.

A test sample of each liquid composition was prepared by extraction of the liquid composition with DMSO media as 100 mg/ml, then dilution of the sample in PBS to a concentration of 5 mg/ml.

A control sample was produced by manually opening green-lipped mussels, extracting and incubating the flesh at 55° C. for 60 minutes then homogenising the flesh followed by extraction with DMSO media as 100 mg/ml, then dilution of the sample in PBS to a concentration of 5 mg/ml.

The results of the test are shown in FIG. 13. The results show that the compositions of Example 2 had a very high level of COX-2 inhibition activity.

COX-2 inhibition activity is linked to anti-inflammatory activity and it is expected that due to the unique structure and properties of the compositions of the invention, the bioactive anti-inflammatory components present in the compositions will have improved bioavailability and therefore good efficacy in the treatment of inflammation conditions.

### EXAMPLE 3

Blue mussels (Mytilus edulis) were processed by adding 60 kg of live whole mussels to a sealable, pressurisable treatment chamber. The chamber was closed and a warming step in the form of a flash steam injection into the chamber at a temperature of 100° C. for a period of 90 seconds was employed. At the same time the chamber was rotated (by external rotation means) for about 5 minutes to achieve an optimum temperature of about 45-50° C. evenly distributed inside the chamber in order to open or gap the mussels and to condition the mussels for the enzyme treatment step. The enzyme treatment comprised application of 6% of an enzyme formulation (based on the total protein amount of between 3-4 kg) comprising a proteolytic enzyme derived from Bacillus species (commercially available as ALCALASE). Alternative enzymes such as ESP153 or ENZIDASE PTX6L or NEUTRASE could have been used. The internal temperature of the chamber was maintained at about 55-60° C. by the initial steam injection. The treatment duration was about 30 minutes. The contents of the chamber were discharged onto a separating screen to remove any residual shells, shell fragments and any other large particles. The remaining liquid composition was filtered and stabilised. It was observed that the structure and stability of the composition was substantially similar to those prepared with green-lipped mussels, indicating that similar compositions and extracts can be produced from other mussel species.

Advantages

The mussel compositions or extracts of the invention have the following potentially realisable advantages:
a) Increased yield of bioactive components
b) Structure of liquid compositions allows for multiple drying options to be used, including spray drying
c) Structure of liquid and dried compositions allows for multiple fractionation, separation and extraction processes to be used to make various finished products
d) The small particle size and stable and uniform nature of the liquid compositions allow for other direct non-thermal sterilization processes, such as Pulsed Electric Field (PEF) or Ultra-High Temperature (UHT), or High Temperature/Short Time (HTST) pasteurization to be used
e) The compositions are highly soluble which improves their ability to be formulated in many applications
f) The compositions have improved sensory attributes including smell, taste and flavour profiles making the compositions suitable for many applications
g) The emulsion-like structure of the compositions having high solubility in aqueous mediums allows for improved bioavailability due to an increased ability to be absorbed into the body
h) Desirable bioactive lipid compounds are naturally protected or encapsulated in the compositions increasing the bioavailability and efficacy of these compounds
i) The compositions are naturally stable and do not require additional non-natural surfactants, co-surfactants or other emulsifying agents or additives for stabilisation.

Variations

It will of course be realised that while the foregoing has been given by way of illustrative example of this invention, all such and other modifications and variations thereto as would be apparent to persons skilled in the art are deemed to fall within the broad scope and ambit of this invention as is hereinbefore described.

While the examples show compositions produced by enzyme hydrolysis methods using certain enzymes, these show some preferred enzyme formulations only. It is envisaged that most commercially available enzymes would be effective in producing the compositions of the invention, and the actual selection of enzymes is determined based on many factors such as the optimum processing temperature and pH of each enzyme, the time it generally takes to obtain a sufficient degree of hydrolysis with each enzyme type, the respective costs and general accessibility of different types of enzymes. In addition, the species of mussel and target substrates of the mussels should be considered as well as the desired end-products and specifications.

In the examples, green-lipped mussels and blue mussels are used. It is expected that similar compositions can be obtained from other species of mussels.

It will also be understood that where a product, method or process as herein described or claimed is sold incomplete, as individual components or as a “kit of parts”, or is carried out as individual or separate steps, that such exploitation will fall within the ambit of the invention.

For purposes of the description hereinafter, the terms “upper”, “lower”, “right”, “left”, “vertical”, “horizontal”, “top”, “bottom”, “lateral”, “longitudinal”, “side”, “front”, “rear” and derivatives thereof shall relate to the invention as it is oriented in the drawing figures. However it is to be understood that the invention may assume various alternative variations, except where expressly specified to the contrary. It is also to be understood that the specific devices illustrated in the attached drawings, and described in the following specification are simply exemplary embodiments of the invention. Hence specific dimensions and other physical characteristics related to the embodiments disclosed herein are not to be considered as limiting.

1-17. (canceled)

18. A whole mussel composition having at least two phases, including at least one hydrophobic phase comprising one or more lipid and/or lipophilic bioactive compounds, and one or more hydrophilic or aqueous phases having one or more bioactive components dispersed or suspended therein.

19. The mussel composition of claim 18 wherein the composition is in the form of a liquid composition comprising an emulsion-like structure wherein the at least one hydrophobic phase comprises one or more lipid and/or lipophilic bioactive compounds, and said one or more lipid and/or lipophilic bioactive compounds are dispersed in said hydrophilic or aqueous phase, and wherein said hydrophilic or aqueous phase has one or more hydrophilic bioactive components dispersed or suspended therein.

20. The mussel composition of claim 18 wherein the hydrophobic phase comprises at least some droplets or globules having a layer surrounding or encapsulating the droplets or globules wherein the lipid or lipophilic bioactive components are located inside the droplets or globules.

21. The mussel composition of claim 18 wherein the composition comprises a mixture of particle sizes, with a mean particle size distribution of between about 100 nm-100 μm, including some micro-particles and/or micro-droplets and/or some nano-particles and/or nano-droplets.

22. The mussel composition of claim 18 wherein the majority of the particles in the composition are micro-particles with sizes in the range of between 100-50,000 nm.

23. The mussel composition of claim 18 wherein the composition comprises at least 5% lipid or lipophilic components in the hydrophobic phase, and at least 75% hydrophilic components in the hydrophilic phase.

24. The mussel composition of claim 18 wherein the composition comprises one or more natural components functioning as emulsifiers, surfactants and/or co-surfactants.

25. The mussel composition of claim 24 wherein the one or more natural components functioning as emulsifiers, surfactants and/or co-surfactants are proteins and/or peptides in the hydrophilic phase, and/or located on the surface of the particles or globules in the hydrophobic phase.

26. The mussel composition of claim 18 wherein the composition is a dried composition.

27. The mussel composition of claim 26 wherein the dried composition when rehydrated in water or aqueous media forms a liquid composition as defined in claim 2.

28. The mussel composition of claim 18 wherein the composition has the properties and/or characteristics of a self-emulsifying composition.

29. A lipid mussel extract produced from a mussel composition of claim 18 by extraction or fractionation and recovery of the hydrophobic phase of the mussel composition.

30. A non-lipid mussel extract produced from a mussel composition of claim 18 by extraction or fractionation and recovery of the hydrophilic phase of the mussel composition.

31. A product comprising a mussel composition as claimed in claim 18.

32. A dried self-emulsifying mussel composition and/or bioactive delivery system derived from mussels, said mussel composition having at least two phases, including at least one hydrophobic phase comprising one or more lipid and/or lipophilic bioactive compounds, and one or more hydrophilic or aqueous phases having one or more bioactive components dispersed or suspended therein, and one or more natural components functioning as surfactants and/or co-surfactants and/or emulsifiers, such that on addition to an aqueous media the composition forms an emulsion-like structure.
